(19) United States
(12) Patent Application Publication
(10) Pub. No.: US 2004/0208788 A1
(43) Pub. Date: Oct. 21, 2004

(54) Polymer micro-cantilevers and their methods of manufacture

(76) Inventor: Jonathan S. Colton, Atlanta, GA (US)

Correspondence Address: ARNALL GOLDEN GREGORY LLP, Suite 2800, 1201 West Peachtree Street, Atlanta, GA, 30309, US

(21) Appl. No.: 10/414,744

(22) Filed: Apr. 15, 2003

(51) Int. Cl. G01N 33/00

(52) U.S. Cl. 422/68.1

(57) ABSTRACT

A micro-cantilever beam is constructed from a thermoplastic polymer material. In a method of designing a micro-cantilever beam, at least one of a plurality of dimensional factors is selected so as to create a beam geometry. A material that the beam will be constructed from is selected so that the material and the beam geometry give rise to a predetermined mechanical figure of merit in the beam. In a method of manufacturing a micro-cantilever beam, a geometry for the micro-cantilever is selected beam. The micro-cantilever beam, according to the geometry, is formed from a thermoplastic material.

### CROSS-REFERENCE TO A RELATED PROVISIONAL PATENT APPLICATION

The present application claims priority on U.S. Provisional Patent Application Serial No. 60/372,468, filed Apr. 15, 2002, the entirety of which is incorporated herein by reference.

### BACKGROUND OF THE INVENTION

1. Field of the Invention

The invention relates to nanotechnology-based sensing systems and, more specifically, to a cantilever beam for use in analysis and atomic force microscopy and the like.

2. Description of the Prior Art

Micro-cantilevers, such as in equipment such as atomic force microscopes (AFM), are giving rise to an emerging sensor platform. The sensing mechanism is straightforward. Molecular adsorption on a resonating cantilever shifts its resonance frequency and changes its surface forces (surface stress). Adsorption onto micro-cantilevers comprised of two chemically different surfaces results in a differential stress between the top and bottom surfaces of the cantilever and induces micro-cantilever bending. Given the current imperatives to develop more sensitive and selective sensors for air-borne and water-borne toxic and pathogenic substances, rapid growth in micro-cantilever-based sensor technology is anticipated.

Micro-cantilever-based detection of airborne components is a growing application of this sensor platform. Mass sensitivities in the picogram to femtogram range are commonplace with optical reflection as the measurement mode. Chemisorption of an analyte into the coating produces a mass increase in the layer as well as a change in its interfacial stress. Thus, high sensitivity detection of individual components can be achieved by monitoring either a deflection or a shift in the resonance frequency of the cantilever. Mixture components can be qualitatively and quantitatively identified using principal component regression analysis.

Currently, the micro-cantilevers used in sensing applications are the same as those used for AFM applications. Commercial AFM cantilevers are typically fabricated from single crystal silicon, silicon dioxide or silicon nitride using conventional silicon micromachining techniques. One side of the cantilever is coated with a thin, reflective gold film to facilitate detection of cantilever deflection by optical deflection techniques. The dimensions and properties are those needed for imaging applications are far from optimal for sensing applications. In addition, the thin metal coating over one side of the cantilever renders the device extremely sensitive to small changes in temperature.

A current goal in medical diagnostic research is establishing the molecular basis of human disease. With this knowledge, the quality and duration of life will be significantly improved through prevention and early diagnosis of disease. A key factor is the development of new drugs and therapeutic monitoring systems. Mapping of the human genome was the first milestone in meeting this challenge. The second milestone lies in determining the structure and function for all proteins for which the genome encodes. While mapping of the genome is near completion, understanding of the structure and function of the proteins for which the genome encodes is in its infancy. To make full advantage of genomic information in eradicating genetically related disease states, technologies for multiplex quantification of proteins are needed, especially for monitoring of the initiation, progression and treatment of disease. Such technologies, for example, could enable profiling of tumor proteomes and improve diagnostics through analysis of constellations of proteins rather than single proteins.

Methods for multiplexed detection of proteins are more challenging than nucleic acid microarray methods due to the inherent instability of proteins, the greater variability in biophysical properties among proteins, and the lack of facile amplification and labeling methods as for nucleic acids. One microarray protein detection scheme attaches probes to a solid surface and modifies the protein analytes with fluorescent tags in order to detect them. Drawbacks of this methodology are background fluorescence and chemical modification with fluorescent tags that can block molecular recognition. The development of label-free technologies for detecting molecular interactions would be particularly advantageous.

Biosensor devices based on nanomechanical motion of micro-cantilevers comprise an emerging sensor platform having far-reaching potential in determining the molecular basis for disease. Molecular adsorption on a resonating micro-cantilever shifts its resonance frequency; the resonance shift is correlated with change in mass of the cantilever. Another more sensitive device detects the change in surface stress upon the interaction of analytes with molecules tethered to one surface of micro-cantilevers possessing two chemically different surfaces. Differential surface stress induces micro-cantilever bending which can be measured with angstrom resolution. High selectivity in response is achievable through incorporation of biomolecular recognition elements into thin film coatings on the cantilever. Numerous micro-cantilevers having probes for a variety of proteins can be located in a single microfluidics cartridge, enabling multiplexed detection without protein tagging.

Micro-cantilever biosensors are expected to have several technical advantages over alternative sensor technologies, including greater sensitivity, less interference with the biochemical reactions and time-phased measurements of binding reactions. Since the surface area of the micro-cantilever sensor is very small (<0.004 mm2) and each molecular binding reaction contributes to the bending force on the micro-cantilever, very little analyte is required to yield a positive test indication. Since the binding reaction itself induces the bending force, intermediate steps or processes do not alter the signal. Furthermore, native molecules (i.e., not altered with a dye or tag) are attached to the micro-cantilever sensor, so there is no distortion of or interference with the desired biochemical reactions. Other advantages include: (1) The screening of multiple receptor molecules in a single fluid cell, the requirement for assaying each and every chemical for possible interaction with each and every receptor or enzyme target of interest is eliminated. With multiple target receptors arrayed in a single fluid cell or incubation chamber, faster and cheaper screening of the ever-growing number of chemicals against the many thousands of cell receptors becomes more likely; (2) Potential high sensitivity; (3) Potential high specificity; and (4) Small sample size requirement translates into less waste of expensive reagents.

Cantilevers for surface probe microscopy, such as atomic force microscopy, currently are typically made from silicon. Most have very sharp tips on the end to allow high-resolution imaging. The traditional method of cantilever fabrication begins with a silicon on insulator (SOI) wafer. This is a time consuming and expensive fabrication process that uses toxic etchants. Preparation of thin cantilevers can be quite challenging. Residual stresses produce bent cantilevers. As a result, there are a limited number of cantilever suppliers and there is little choice in cantilever geometry, flexibility and material and the resulting cantilevers are relatively costly. The most economical cantilevers are sold in wafer form (between 400-600 cantilevers per wafer). As silicon is a brittle material, one often breaks a handful of tips while trying to set up an experiment, hence raising the actual cost. Commercial silicon cantilevers are quite stiff, typically having a spring constants in the range from 0.01 to 10 N/m. This stiffness is inadequate for certain sensing applications.

For optimal performance, the stiffness of surface probe microscopy (SPM) cantilevers should be matched to the forces being measured. If a beam is too stiff, it will not deflect measurably for small forces. If the beam is too flexible, it will deflect too much or non-linearly for a large force. The stiffness of any cantilever beam results from the inherent properties of the material (for example, the Young's modulus) and the geometry of the beam. The geometry is somewhat set by the commercially available SPM and AFM equipment. For the majority of existing SPM systems, cantilever width is approximately 50 μm to maximize laser light reflection and minimize optical interference. Cantilever lengths typically range from 75 to 300 μm. The thickness is limited by processing conditions, and also by the interplay of the inherent stiffness of the material and the desired beam stiffness. Short cantilevers have recently become available. These were designed to facilitate high speed imaging and single molecule mechanical testing. Use of these requires a specialized optical detection system for measuring cantilever deflection and resonance.

The typical processing steps for fabrication of commercial cantilevers from silicon wafers include: (1) Grow 1 micron SiO2 (both sides); (2) Grow 1 micron Si3N4 (both sides); (3) Pattern tip (e.g., plasma etch nitride); (4) Define tip using a dry etch; (5) Pattern and etch the cantilever using a dry, anisotropic etch; (6) Protect the tip side with polyimide; (7) Pattern and etch backside (dry etch, wet etch); (8) Remove large Si underlayer with a wet etch; and (9) Etch the middle oxide stop layer using a buffered oxide etch.

For rectangular cantilevers, the relationship between its length, l, width, w, and thickness, t, to its stiffness (spring constant, k) is given by:

$$k = \frac{E w {t}^{3}}{4 {l}^{3}}$$

where E is the Young's modulus of the material from which it is fabricated. The relationship between the cantilever's dimension and its resonance frequency, ν₀, is given by:

$${v}_{0} = \sqrt{\frac{E {t}^{2}}{4 {π}^{2} {l}^{2} ρ}}$$

where ρ is the density of the material from which the cantilever is fabricated. Thus, the usual approach to tuning the spring constant and resonance frequency of the cantilever lies in varying its length and thickness. For sensor or soft sample imaging applications, this approach is not optimal. An additional parameter, the minimum detectable force, Fₘᵢₙ must also be taken into consideration. This parameter depends on the cantilever dimensions (through k and ν₀) as well as its quality factor and viscous damping factor:

$${F}_{min} = \sqrt{\frac{2 {K}_{B} T B k}{Q {v}_{0} π}}$$

where K_{B} is the Boltzmann constant, T is the temperature in Kelvin, Q is the quality factor, B is the viscous damping factor. The traditional approach for increasing flexibility was to fabricate longer, thinner cantilevers.

Molecular interactions at or near the cantilever surface may produce an increase in cantilever mass or surface stress. Measurement of the rate and extent of these interactions are thus attainable by recording the cantilever's resonance frequency or deflection over time. If one assumes that the stress is uniformly distributed over the entire cantilever and that the response to this stress causes curvilinear deformation along its length, then the differential surface stress, Δs, is given by:

Δs=Et²[6R(1−ν)]^{-1/2}

where R is the radius of curvature and v is the Poisson's ratio for the material used in cantilever manufacture. In liquid media, viscous damping decreases the amplitude of resonance, diminishing the sensitivity for detection of molecular interactions. With optical deflection techniques, measurement of cantilever deflection can be made with angstrom level precision and, when flexible cantilevers are used, with greater sensitivity. Similarly, measurement of frequency shifts in cantilever resonance can be made with parts per billion level precision.

Cantilevers also deflect in response to changes in temperature, magnetic field strength (if coated with a magnetic material), electrostatic charge, and fluid flow. Thus, attainment of optimal sensitivity to molecular interactions at its surface requires careful design of the cantilever, reader, and sample delivery system as well as environmental control. Secondly, deflection occurs only when a sufficient number of molecular interactions result in a change in surface stress sufficient to overcome the resistance to bending (spring constant). For biosensor applications the number of molecular interactions is determined by the surface area of the active side of the cantilever, the number of covalently attached probe molecules, and the entropic impact of this interaction. Thirdly, the temporal response of cantilever deflection is limited by the rate of mass transport of the target to the probe. Transport of material to the cantilever surface can be achieved by convection, migration (or polarization), and diffusion. Minimization of sample volumes needed for analysis and cantilever response to changes in fluid flow rate (i.e., convection) and migration (i.e., electrostatics), necessitates small cell volumes and diffusion-based transport. Thus, optimization of the analytical sensitivity for micro-cantilever-based immunoassays requires careful consideration of the following interdependent factors: (1) Cantilever spring constant; (2) Surface area of the active element; (3) Sample and cell volumes; (4) Spatial distribution and orientation of the probe on the surface; (5) Affinity of all surfaces for non-specific binding of target and matrix components; (6) Optical gain; (7) Positional sensitivity of the reader.

Photopolymer-based SPM cantilevers have been previously proposed. These were developed for internal, laboratory use and have not been produced in large, economic and commercial quantities. All were produced using microelectronics manufacturing techniques, requiring expensive tooling housed in a clean room environment. In one example, cantilevers were produced from an epoxy-based photopolymer, a photoresist material of the type used in microelectronics processing that is quite brittle and that may not be suitable for many sensing applications. Photopolymers react (cure) when exposed to light, so one creates the required cantilever shape by exposing the photopolymer to patterned light. The tips were placed on the cantilever using electron beam deposition (EBD), which is not well suited for mass production. Reactive ion etching and photopolymer have been used to create cantilevers and tips. In another example polymer cantilevers were produced from SU-8, a epoxy-based photopolymer used as a photoresist in microelectronics manufacturing. A silicon mold, produced using traditional isotropic and anisotropic etching techniques, could be cleaned and reused. The cantilever, tip and chip was fabricated by sequentially spin coating SU-8 onto the wafer, photolithographically crosslinking the polymer followed by rinsing and thermal curing of the crosslinked SU-8. A major challenge was control of the cantilever thickness, a critical parameter in determining cantilever stiffness.

In another example cantilevers were formed from fluoropolymers to produce cantilevers and structures for bio-micro electronic mechanical systems (MEMS). The goal was to produce cantilevers that can be biochemically functionalized for AFM. These were manufactured by ion beam etching, a complicated and expensive process that is not amenable to mass production. To date, polymer cantilevers have been produced using techniques that facilitate formation of sharp tips on their ends for imaging applications. For most sensing applications, a tip is superfluous. Also, the methods of fabrication inherently limit the number of polymeric cantilevers that can be produced at one time. It is clear that more robust, more economic cantilevers with variable mechanical properties and improved biocompatibility would be a boon to the field of scanning probe microscopy and micro-cantilever sensors.

Injection molding is by far the most used to mass-produce complex, three dimensional thermoplastic polymer products. Micromolding, the molding of polymer parts with dimensions on the order of 100s of μm and features on the order of 5-10 μm, is just now coming into commercial use. Injection molding machines capable of producing such parts are now commercially available. This technique has the potential for extremely high throughput. In traditional injection molding, cycle times are on the order of tens of seconds, leading to a time per part of seconds or less for multiple cavity molds. The most complicated have 144 cavities, but most have tens of cavities. As a result of these mass production techniques, polymer parts are quite cheap, with a rule of thumb being two to three times the cost of the material used to make the part. In addition, injection molding is a very well understood and controllable process. Therefore it repeatably produces high quality parts, at a very high level of performance.

In the fabrication of microfluidic devices, such as flow chambers for micro-electrophoresis, hot embossing is a commonly used technique. This technique presses a hot mold (which is a negative of the part desired) into a polymer sheet. Common polymer materials used include PMMA and polycarbonate (PC). Polystyrene is a thermoplastic polymer with a long history of use in medicine, biochemistry and molecular biology. Plastic parts (e.g., microtiter plates and Petri dishes) are made from polystyrene by heating it to soften it, forcing into shape by injecting it into a mold, cooling it to harden, and then removing it from a mold. There are no chemical reactions, hence the process is quite clean and environmentally friendly. Mass production of polymer products from polystyrene is widely practiced and very well characterized polymer processing field.

Therefore, there is a need for a micro-cantilever with controlled response properties.

There is also a need for a micro-cantilever that can be mass-produced inexpensively.

### SUMMARY OF THE INVENTION

The disadvantages of the prior art are overcome by the present invention, which, in one aspect, is an apparatus that includes a micro-cantilever beam constructed from a material including a thermoplastic polymer material.

In another aspect, the invention is a device for quantifying at least first substance and a second substance in an analyte. The device includes an array of thermoplastic micro-cantilever beams, including a base and at least a first micro-cantilever beam extending outwardly from the base. A second micro-cantilever beam is spaced apart from the first micro-cantilever beam, extends outwardly from the base. A first reactive treatment is applied to the first micro-cantilever beam. The first reactive treatment causes the first micro-cantilever beam to exhibit a first physical property in a first manner when the reactive treatment has not reacted with a target substance and causes the micro-cantilever beam to exhibit the first physical property in a second manner, different from the first manner, when the reactive treatment has reacted with the first substance. A second reactive treatment, different from the first reactive treatment, is applied to the second micro-cantilever beam. The second reactive treatment causes the second micro-cantilever beam to exhibit a second physical property in a first manner when the second reactive treatment has not reacted with the second substance and causes the second micro-cantilever beam to exhibit the second physical property in a second manner, different from the first manner, when the second reactive treatment has reacted with the second substance.

In another aspect, the invention is a method of designing a micro-cantilever beam in which at least one of a plurality of dimensional factors is selected so as to create a beam geometry. A material that the beam will be constructed from is selected so that the material and the beam geometry give rise to a predetermined mechanical figure of merit in the beam.

In yet another aspect, the invention is a method of manufacturing a micro-cantilever beam. A geometry for the micro-cantilever is selected beam. The micro-cantilever beam, according to the geometry, is formed from a thermoplastic material.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a plan view of one embodiment of a micro-cantilever and a base, according to the invention.

FIG. 2 is a plan view of a mold according to one embodiment of the invention.

FIG. 3A is a side cross-sectional view of an injection molding machine and a mold prior to injection of a material into the mold.

FIG. 3B is a side cross-sectional view of an injection molding machine and a mold after injection of a material into the mold

FIG. 4A is a schematic diagram of a reactive treatment applied to a micro-cantilever beam prior to reaction with a reactant.

FIG. 4B is a schematic diagram of a reactive treatment applied to a micro-cantilever beam after reaction with a reactant.

FIG. 5 is a schematic diagram of a micro-cantilever beam and a deflection detector.

FIG. 6 is a block diagram of an analysis system according to one embodiment of the invention.

FIG. 7 is a side view of a micro-cantilever beam with a tip.

FIG. 8 is a side view of a micro-cantilever beam that including an optical channel.

FIG. 9 is a side view of a micro-cantilever beam including reinforcement.

FIG. 10 is a plan view of a micro-cantilever beam having a specific geometry.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Unless otherwise specified herein, the drawings are not necessarily drawn to scale.

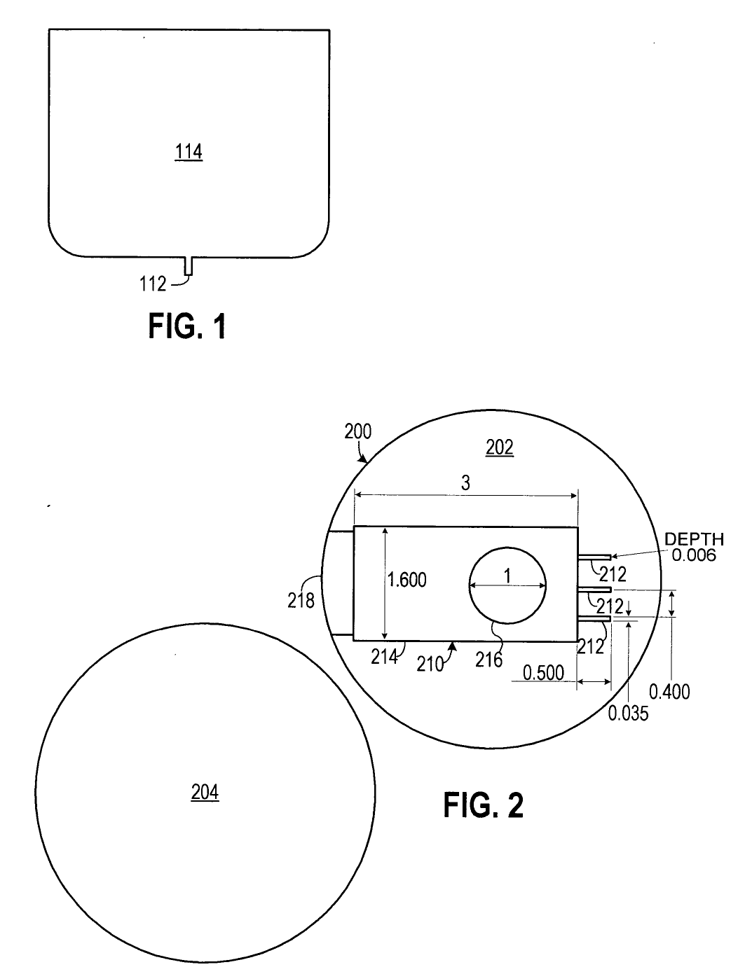

As shown in FIG. 1, a micro-cantilever beam 112, according to the invention, is made from a thermoplastic polymer material. The micro-cantilever beam 112 could extend outwardly from a base 114 that can also be made from the thermoplastic material. The micro-cantilever beam 112 could be manufactured through one of several processes employed in micro-fabrication, including: injection molding, shaping a fiber, cutting a pre-shaped fiber to a predetermined length, casting by placing an uncured polymer into a mold and allowing the polymer to cure, and cutting the micro-cantilever beam 112 from a sheet of thermoplastic of a suitable thickness. The thermoplastic resin could include: polystyrene, polypropylene, polyethylene, acrylonitrile butadiene styrene, polycarbonate, PMMA, polyester, or polyamide, or a combination thereof. Using a thermoplastic in manufacturing the micro-cantilever beam 112 offers the advantage of decreased cost of manufacturing (the cost of manufacturing en masse being on the order of several times the cost of the raw thermoplastic material being used) and the added utility of being able to affix reactants directly to the micro-cantilever beam 112 without applying another material, such as gold, first.

Two alternate techniques that may be used for mass production of a micro-cantilever beam include hot embossing, and laser ablation. Hot embossing is a process whereby a die in the shape of the part is heated and stamped into a polymer film. This process is similar to coining in metals, in that very little material is displaced and very fine and accurate features can be produced. A die could be manufactured in the shape of a comb, using any number of manufacturing techniques, such as micro-EDM, electrochemical machining, laser machining, or reactive ion etching. This die then would then be stamped into a very thin film of the polymer of interest, forming the cantilevers by melting away the unwanted material. Laser ablation could be used to directly form the cantilever tips from sheets of polymer. A laser could be directed to remove (melt) material and hence form the tips. One advantage of this process is the width of the laser beam only needs to be on the order of the size of the gaps between the cantilevers, not the cantilever it self. An eximer laser would be suitable for this process.

As shown in FIG. 2, (all dimensions in FIG. 2 are given in millimeters) a mold 200 may be used to manufacture the micro-cantilever beam. The mold 200 shown in FIG. 2 could be used to manufacture a plurality of micro-cantilever beams simultaneously. The mold 200 could include a plate member 202, typically made from a suitable metal, such as steel. A cavity 210, defined in the plate member 202, includes one or more micro-cantilever beam-shaped cavities 212 extending outwardly from a base cavity 214. An injection port 218 allows material to be injected into the cavity 210 and a hole for a knock-out pin 216 facilitates removal of the micro-cantilever beams from the mold 200. The cavity may 214 be formed in the plate using one of several methods, including: micro-electrical discharge machining; LIGA; etching; machining; laser ablation; or electro-chemical machining. A base member 204 is placed against the plate member 202 form the bottom of the cavity 210.

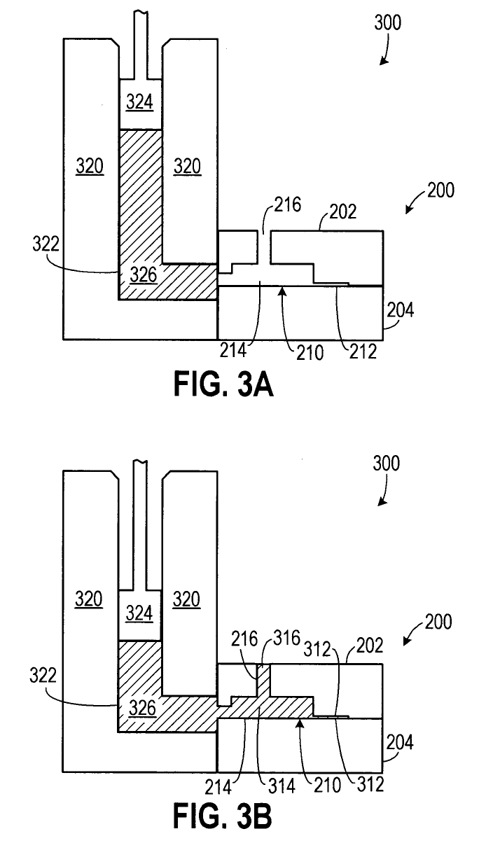

An injection molding system 300 is shown in FIGS. 3A and 3B. In such a system, the mold 200 is placed against an injector 320 that includes a thermoplastic material 326 well 322 and a piston 324 for extruding the thermoplastic 326 from the well 322 into the cavity 210 defined by the mold 200. Typically, a heater (not shown) is included with the injection molding system 300 to melt the thermoplastic material 326 and heat the mold 200.

Injection molding may be accomplished through the use of a NanoMolding machine (such as model Sesame 080, from Murray Engineering, Buffalo Grove, Ill.). Such a machine is capable of injecting milligrams of material at a time. The NanoMolding machine is a dual plunger system. A vertical plunger melts the thermoplastic pellets by forcing them through a heated capillary. This creates the shot in front of the injection plunger. The injection plunger then forces the plastic into the empty cavity, thereby producing the part. The part cools and is removed.

Injection molding allows the making of many identical cantilevers. Typical injection molds for commercial applications contain multiple cavities. This allows for a higher utilization of the injection molding machine and mold.

In one example of a cantilever beam manufactured according to the invention, cantilever beams without tips had initial dimensions as follows: 500 μm long, 50 μm wide and 5 to 10 μm thick. This resulted in cantilevers with moduli in the 5-10 mN/m range.

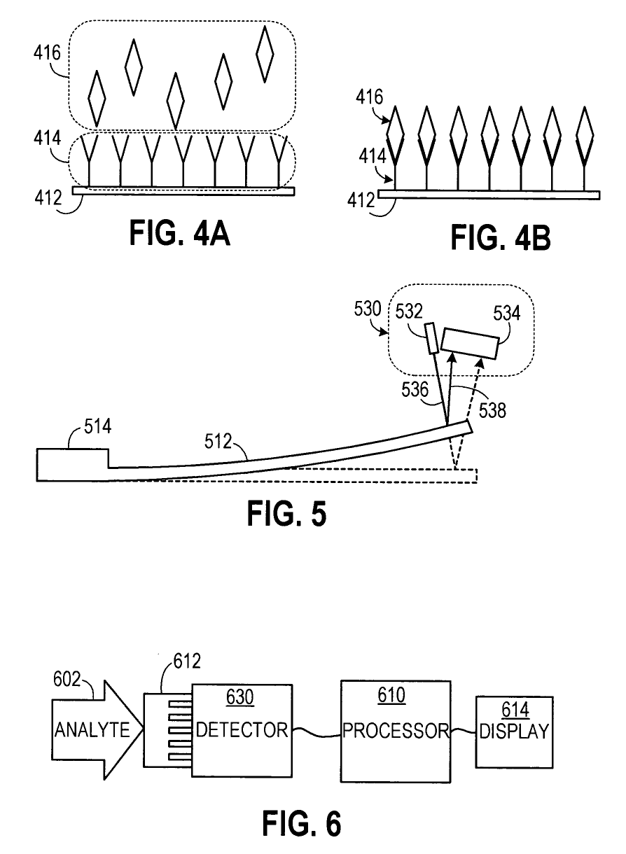

Micro-cantilever beams, according to the invention, could be employed in analysis and sensing systems, to perform such tasks as bioassay and molecular assay analysis. As shown in FIGS. 4A and 4B, in one embodiment, the micro-cantilever beam 412 could include a reactive treatment 414 that is applied to a selected side of the micro-cantilever beam 412. The reactive treatment 414 will cause the micro-cantilever beam 412 to exhibit a predetermined change in a physical property (such as deflection or frequency response) in a first manner when the reactive treatment has not reacted with a selected substance 416 (as shown in FIG. 4A). The reactive treatment 414 causes the micro-cantilever beam 412 to exhibit the physical property in a second manner, different from the first manner, when the reactive treatment 414 has reacted with the selected substance 416.

The composition of the reactive treatment 414 would depend upon the substance being sensed. For example, in an imuno-assay, the reactive treatment 414 could include an antigen that is receptive to an antibody. In an assay of an infectant, the reactive treatment 414 could include an antibody that is receptive to an antigen or some other protein associated with the infectant. Similarly, the reactive treatment 414 could include a treatment used in molecular recognition, a treatment used in biological recognition, a treatment used in bio-molecular recognition, or one of many other types of reactive treatments used to recognize substances.

While not shown, the micro-cantilever beam 412 could include two different reactive treatments (or the same reactive treatment is different concentrations) applied to opposite sides of the micro-cantilever beam 412 to derive information, for example, about the proportion of one substance to another substance.

The physical properties associated with the micro-cantilever beam 412 could include deflection of the micro-cantilever beam 412 from a non-reactive state and a change in frequency response.

Sensing the state of the micro-cantilever beam 512 is shown in FIG. 5. One end including a beam holder 514 of the micro-cantilever beam 512 is secured to a stable platform (not shown), while the other end is allowed to move freely in at least one axis. A beam state detector 530 includes a light source 523 that generates a laser beam 536 that reflects off of a predetermined spot of the micro-cantilever beam 512. The reflected beam 538 then is sensed by a photo-detector 534. The amount of deflection could be determined by measuring the displacement of the reflected beam 538 or through the use of interferometry. While FIG. 5 shows one example of a micro-cantilever beam state sensor, many other types of micro-cantilever beam state sensors are available and would be suitable for use with embodiments of the invention.

A system for detecting several substances in an analyte 602 (or for quantifying a concentration of a single substance) is shown in FIG. 6. The system includes an array of micro-cantilever beams 612, around which the analyte 602 is passed. Each micro-cantilever beam of the array 612 could be treated with a different reactive treatment to detect different substances in the analyte 602 or could be treated with the same reactive treatment in differing densities to detect a concentration of a single substance in the analyte 602. A detector 630 senses the state of the micro-cantilever beams 612, once the system is in steady state, and transmits the state information to a processor 610. The processor 610 determines the presence or concentrations of the substances being assayed and the resulting information is output to a user interface, such as a display 614.

In one specific embodiment, the detector for multi-micro-cantilever could include 670 nm diode lasers, a fluid cell and quadrant photodiode position-sensing devices (PSD) (available from Pacific Sensor Inc.). Laser light is reflected off the reflective side of the cantilever onto the PSD. The voltage output of the PSD is proportional to the magnitude of cantilever deflection whereas the sign is indicative of the direction of bending (up or down with respect to a metal-coated side). The voltage output of each PSD is amplified (each PSD is mounted on its own amplifier card) and sent to a Signal Analyzer (such as a Model 785 Dynamic available from Stanford Research Systems). Frequency shifts in cantilever resonance may be measured by taking repeated Fourier transforms of the time dependent PSD. Frequency versus time data may then be downloaded to a computer for display. Cantilever deflection measurements may be recorded by taking the voltage output of each PSD (normalized for changes in diode laser output) as a function of time by downloading this data directly to a laboratory computer for display.

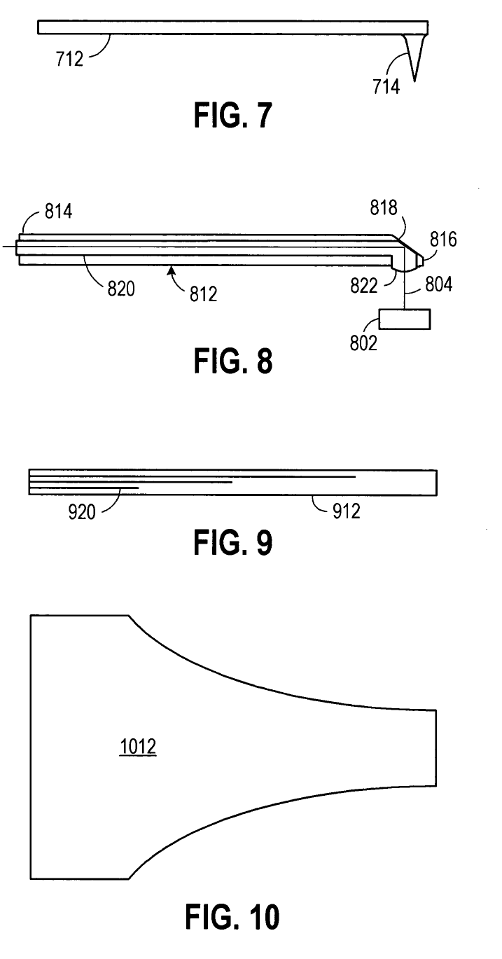

A tip 714 may be added to the micro-cantilever beam 712, as shown in FIG. 7. Addition of a tip 714 could be performed in one of several ways, including: molding the tip 714 onto the micro-cantilever beam 712 as part of a molding process, thereby making an integrated beam and tip unit; gluing the tip 714 to the beam 712; heating the tip 714 and melting it into the beam 712; or one of many other methods of attachment. Use of the tip 714 could allow the micro-cantilever beam 712 to be used in such applications as atomic force microscopy (AFM) and dip pen lithography. The tip 714 could be a silicon AFM-type tip, a plastic tip, a carbon nanotube or one of many other types of tip.

A micro-cantilever beam 812 according to the invention, as shown in FIG. 8, could include an embedded optical channel 820 extending from a proximal end 814 to a distal end 816 of the beam 812. The optical channel 820 could terminate in a lens 822 and include a reflector 818. The optical channel could be used to guide an electromagnetic beam 804 to and from a surface 802 being imaged. The optical channel 820 could also be used to deliver a light beam to a preselected spot for such applications as micro-laser ablation. A change in the geometry of the optical channel 820, indicating a deflection of the micro-cantilever beam 812, could be sensed through interferometry.

As shown in FIG. 9, an additive, such as a reinforcing agent, could be added to the thermoplastic material of the micro-cantilever beam 912 so as to modify a mechanical figure of merit of the micro-cantilever beam 912. For example strips 920 of a different substance than the thermoplastic material could be embedded in the micro-cantilever beam 912 along one or more preselected planes to modify stiffness and linearity of response. Also, other additives could be used, including: nanotubes, nanoparticles, nanofibers, microtubes, microparticles, microfibers, tubes, particles, or fibers. Additives could also be added to modify other physical properties, including: electrical conductivity; frequency response; minimum force required to deflect and many other properties that facilitate use of the invention in specific applications.

As shown in FIG. 10, alternate geometries of the micro-cantilever beam 1012 may be used to further refine the physical characteristics of the micro-cantilever beam 1012. The geometry could include several dimensional factors, such as: length, width, height and shape. For example, a geometry that gives the micro-cantilever beam 1012 a linear response to a linear force applied thereto could be selected. The specific thermoplastic material, possibly in combination with reinforcing agents, could be selected to tune the physical behavior, e.g., stiffness, frequency response, linearity of deflection, minimum force required to deflect the beam, of the micro-cantilever beam 1012.

Young's modulus of polystyrene (approximately 3 GPa) makes it ideal for use as a SPM cantilever probe. Other polymers, such as polymethylmethacrylate (PMMA), polycarbonate (PC), polyethylene (PE) and polypropylene (PP) are also thermoplastics, having widely varying moduli (stiffness) and bio-chemical compatibility. As a result of their different moduli, varying stiffness cantilevers could be manufactured with the same geometry. The latter is important so that the cantilevers will be compatible with commercial SPM equipment (will fit them without modification). Another way to vary the modulus of a polymer material is to add reinforcements in the form of particles or fibers, hence forming a composite. Nano-clays or nano-fibers could be added in very small levels to polystyrene to change its mechanical properties, without affecting its bioadhesion. As a result, one could tune the mechanical properties of a cantilever, without changing its geometry, which is important for assuring its compatibility with existing SPM equipment.

One can design to the desired stiffness of the cantilever by varying the basic geometry (length, width, thickness), of course within limits circumscribed by compatibility with existing equipment. Once the mold has been fabricated to specified dimensions, the spring constant of the cantilever can be tuned by varying the polymer composition. This manipulation would produce tunable spring constants, and hence allow one to vary its sensitivity as a sensor. The addition of fillers, such as nano-scale particles (clays), fillers (carbon, glass, ceramic, polymeric, metallic), and fibers (carbon, glass, carbon nanotubes, metallic, ceramic), hence forming composites and functionally gradient materials, allows the stiffness of the base material to be varied by orders of magnitude, without changing the chemical properties of the surface of the cantilever. The geometry of the cantilever also could be varied to accomplish tunable spring constants. For example, the beam's width could taper towards it end, which would give interesting mechanical response, such as a constant spring force regardless of deflection.

The following table relates various polymer materials to other cantilever beam physical properties:

| | | Cantilever | Cantilever |
|---|---|---|---|
| | | stiffness | stiffness |
| | Young's | (50 μm wide) | (35 μm wide) |
| Material | modulus (GPa) | (mN/m) | (mN/m) |
| polystyrene | 3.0 | 37.5 | 26.3 |
| polycarbonate | 2.4 | 30 | 21 |
| polymethylmetha- | 2.6 | 32.5 | 22.8 |
| crylate | | | |
| polyethylene | 1.1 | 13.8 | 9.7 |
| polyproplylene | 1.3 | 16.3 | 11.4 |
| ABS | 2.4 | 30 | 21 |
| polytetrafluoroethylene | 0.55 | 6.9 | 4.8 |
| nylon 6 with 1.6 vol % | 2.1 (1.1) | 26.3 (13.8) | 18.4 (9.7) |
| nanoclay filler (neat | | | |
| nylon 6) | | | |

As one can see, a wide variety of stiffness can be obtained by changing the material, the dimensions or both. These materials also cover a wide range of reactivity with biological materials, allowing one to select.

The above described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

What is claimed is:

1. An apparatus, comprising a micro-cantilever beam constructed from a material including a thermoplastic polymer material.

2. The apparatus of claim 1, further comprising a reinforcing agent that is added to the thermoplastic polymer material to modify a mechanical figure of merit for the micro-cantilever beam.

3. The apparatus of claim 2, wherein the reinforcing agent comprises at least one metal strip placed along a preselected plane of the micro-cantilever beam.

4. The apparatus of claim 2, wherein the reinforcing agent comprises a plurality of nanotubes added to the thermoplastic polymer material.

5. The apparatus of claim 2, wherein the reinforcing agent comprises a plurality of nanoparticles added to the thermoplastic polymer material.

6. The apparatus of claim 2, wherein the reinforcing agent comprises a plurality of nanofibers added to the thermoplastic polymer material.

7. The apparatus of claim 2, wherein the reinforcing agent comprises a plurality of microtubes added to the thermoplastic polymer material.

8. The apparatus of claim 2, wherein the reinforcing agent comprises a plurality of microparticles added to the thermoplastic polymer material.

9. The apparatus of claim 2, wherein the reinforcing agent comprises a plurality of microfibers added to the thermoplastic polymer material.

10. The apparatus of claim 2, wherein the reinforcing agent comprises a plurality of tubes added to the thermoplastic polymer material.

11. The apparatus of claim 2, wherein the reinforcing agent comprises a plurality of particles added to the thermoplastic polymer material.

12. The apparatus of claim 2, wherein the reinforcing agent comprises a plurality of fibers added to the thermoplastic polymer material.

13. The apparatus of claim 1, wherein the micro-cantilever beam comprises a plurality of sides, the micro-cantilever beam further comprising a reactive treatment applied to at least one side of the micro-cantilever beam, the reactive treatment causing the micro-cantilever beam to exhibit a predetermined physical property in a first manner when the reactive treatment has not reacted with a selected substance and causing the micro-cantilever beam to exhibit the predetermined physical property in a second manner, different from the first manner, when the reactive treatment has reacted with the selected substance.

14. The apparatus of claim 13, wherein the reactive treatment comprises an antibody.

15. The apparatus of claim 13, wherein the reactive treatment comprises a treatment used in molecular recognition.

16. The apparatus of claim 13, wherein the reactive treatment comprises a treatment used in biological recognition.

17. The apparatus of claim 13, wherein the reactive treatment comprises a treatment used in bio-molecular recognition.

18. The apparatus of claim 13, wherein the first physical property comprises a first deflection and the second physical property comprises a second deflection, different from the first deflection.

19. The apparatus of claim 13, wherein the first physical property comprises a first frequency response and the second physical property comprises a second frequency response different from the first frequency response.

20. The apparatus of claim 1, wherein the thermoplastic polymer material comprises polystyrene.

21. The apparatus of claim 1, wherein the thermoplastic polymer material comprises polypropylene.

22. The apparatus of claim 1, wherein the thermoplastic polymer material comprises polyethylene.

23. The apparatus of claim 1, wherein the thermoplastic polymer material comprises acrylonitrile butadiene styrene.

24. The apparatus of claim 1, wherein the thermoplastic polymer material comprises polycarbonate.

25. The apparatus of claim 1, wherein the thermoplastic polymer material comprises PMMA.

26. The apparatus of claim 1, wherein the thermoplastic polymer material comprises polyester.

27. The apparatus of claim 1, wherein the thermoplastic polymer material comprises polyamide.

28. The apparatus of claim 1, wherein the micro-cantilever has a proximal end, an opposite distal end and an elongated dimension there between, further comprising a lens affixed to the distal end.

29. The apparatus of claim 1, wherein the micro-cantilever has a proximal end, an opposite distal end and an elongated dimension there between, further comprising an optical channel between the proximal end and the distal end that is capable of channeling electromagnetic radiation from the distal end to the proximal end.

30. The apparatus of claim 1, wherein the micro-cantilever has a proximal end, an opposite distal end and an elongated dimension there between, further comprising:
a. a lens affixed to the distal end; and
b. an optical channel between the proximal end and the distal end that is capable of channeling electromagnetic radiation from the lens to the proximal end.

31. A sensing apparatus, comprising:
a. a base; and
b. a micro-cantilever beam, extending outwardly from the base, constructed from a material including a thermoplastic polymer material.

32. A micro-cantilever beam unit, comprising:
a. a base portion constructed from a thermoplastic polymer material;
b. an elongated member extending outwardly from the base portion and constructed from a material including a thermoplastic polymer material, the elongated member having an elongated dimension that is less than 0.6 mm.

33. A sensing apparatus, comprising:
a. a base;
b. a micro-cantilever beam, extending outwardly from the base, constructed from a material including a thermoplastic polymer material, the micro-cantilever beam having a distal end opposite the base; and
c. a tip extending downwardly from the distal end.

34. A device for quantifying at least first substance and a second substance in an analyte, the device comprising:
a. a base;
b. at least a first thermoplastic micro-cantilever beam extending outwardly from the base; and
c. at least a second thermoplastic micro-cantilever beam, spaced apart from the first micro-cantilever beam, extending outwardly from the base,
the first thermoplastic micro-cantilever beam and the second thermoplastic micro-cantilever beam forming at least part of an array of thermoplastic micro-cantilever beams.

35. The device of claim 34, further comprising:
a. a first reactive treatment applied to the first micro-cantilever beam, the first reactive treatment causing the first micro-cantilever beam to exhibit a first physical property in a first manner when the reactive treatment has not reacted with a target substance and causing the micro-cantilever beam to exhibit the first physical property in a second manner, different from the first manner, when the reactive treatment has reacted with the first substance; and
b. a second reactive treatment, different from the first reactive treatment, applied to the second micro-cantilever beam, the second reactive treatment causing the second micro-cantilever beam to exhibit a second physical property in a first manner when the second reactive treatment has not reacted with the second substance and causing the second micro-cantilever beam to exhibit the second physical property in a second manner, different from the first manner, when the second reactive treatment has reacted with the second substance.

36. The device of claim 35, further comprising:
a. a cantilever beam state detection circuit that detects a state of the first physical property to determine if the first substance is present in the analyte, and that detects the state of the second physical property to determine if the second substance is present in the analyte; and
b. an indicator circuit, responsive to the cantilever beam state detection circuit, that indicates if either the first substance is present in the analyte or if the second substance is present in the analyte.

37. The device of claim 35, wherein the device indicates a concentration of at least one of the first substance or the second substance.

38. The device of claim 35, wherein the first physical property comprises a first deflection and the second physical property comprises a second deflection, different from the first deflection.

39. The device of claim 35, wherein the first physical property comprises a first frequency response and the second physical property comprises a second frequency response different from the first frequency response.

40. The device of claim 35, wherein the first reactive treatment comprises a first antibody and wherein the second reactive treatment comprises a second antibody.

41. The device of claim 35, wherein the first reactive treatment comprises a first reactant and wherein the second reactive treatment comprises a second reactant.

42. A method of designing a micro-cantilever beam, comprising the steps of:
a. selecting at least one of a plurality of dimensional factors so as to create a beam geometry;
b. selecting a material that the beam will be constructed from so that the material and the beam geometry give rise to a predetermined mechanical figure of merit in the beam.

43. The method of claim 42, wherein the mechanical figure of merit comprises stiffness.

44. The method of claim 42, wherein the mechanical figure of merit comprises frequency response.

45. The method of claim 42, wherein the mechanical figure of merit comprises linearity of deflection.

46. The method of claim 42, wherein the mechanical figure of merit comprises minimum force required to deflect the beam.

47. The method of claim 42, wherein the dimensional factors include length, width, height and shape.

48. A method of manufacturing a micro-cantilever beam, comprising the steps of:
a. selecting a geometry for the micro-cantilever beam; and
b. forming the micro-cantilever beam, according to the geometry, from a thermoplastic material.

49. The method of claim 48, wherein the forming step comprises:
a. creating a mold defining a cavity, a portion of the cavity having a shape and size corresponding to a desired shape and size of a micro-cantilever;
b. heating a thermoplastic polymer material until the thermoplastic polymer material enters a substantially soft phase;
c. injecting the thermoplastic polymer material in the soft phase into the cavity, thereby creating a thermoplastic polymer material cast of the mold;
d. allowing the thermoplastic polymer material cast to cool until the thermoplastic polymer material enters a substantially non-soft phase; and
e. removing the thermoplastic polymer material cast from the cavity.

50. The method of claim 49, wherein the mold creating step comprises the step of removing material from a plate using micro-electrical discharge machining to create the cavity.

51. The method of claim 49, wherein the mold creating step comprises the step of removing material from a plate using LIGA to create the cavity.

52. The method of claim 49, wherein the mold creating step comprises the step of removing material from a plate using etching to create the cavity.

53. The method of claim 49, wherein the mold creating step comprises the step of removing material from a plate using machining to create the cavity.

54. The method of claim 49, wherein the mold creating step comprises the step of removing material from a plate using laser ablation to create the cavity.

55. The method of claim 49, wherein the mold creating step comprises the step of removing material from a plate using electro-chemical machining to create the cavity.

56. The method of claim 49, wherein the micro-cantilever beam has an elongated dimension and a distal end, the method further comprising the step of affixing a sensing tip to the micro-cantilever beam adjacent the distal end.

57. The method of claim 48, wherein the forming step comprises shaping a fiber to form the micro-cantilever beam.

58. The method of claim 48, wherein the forming step comprises cutting a thermoplastic fiber to a predetermined length to form the micro-cantilever beam.

59. The method of claim 48, wherein the forming step comprises casting a thermoplastic to form the micro-cantilever beam.
